(19) United States
(12) Patent Application Publication
(10) Pub. No.: US 2014/0135681 A1
(43) Pub. Date: May 15, 2014

(54) INSTRUMENT AND METHOD FOR ULTRASOUND MEDIATED DRUG DELIVERY

(71) Applicant: Surf Technology AS, Trondheim (NO)

(72) Inventor: Bjorn A. J. ANGELSEN, TRONDHEIM (NO)

(73) Assignee: Surf Technology AS, Trondheim (NO)

(21) Appl. No.: 14/018,990

(22) Filed: Sep. 5, 2013

Related U.S. Application Data
(60) Provisional application No. 61/697,013, filed on Sep. 5, 2012.

(30) Foreign Application Priority Data
May 13, 2013 (GB) 1308579.0

(51) Int. Cl. A61M 37/00

(52) U.S. Cl. CPC A61M 37/0092
USPC 604/22

(57) ABSTRACT

A method and instrumentation for ultrasound mediated delivery of drugs to diseased tissue use ultrasound beams with frequency and focusing that provides an ultrasound radiation force acting on the drug and surrounding fluid, which produces a convection of drugs and compensates for the lack of a pressure gradient. To manipulate drug encapsulations and also stimulate transport of drugs across biological membranes, like the cell membrane or the blood brain barrier, the method and instrumentation use low frequency beams with high mechanical index. The method and instrumentation additionally uses ultrasound heating of the tissue to increase blood flow and manipulate thermally sensitive particles.

### CROSS-REFERENCE TO RELATED APPLICATIONS

This application claims the priority of U.S. 61/697,013 filed Sep. 5, 2012 and GB1308579.0 filed May 13, 2013, the priority of both applications is hereby claimed and both applications are incorporated by reference herein.

### FIELD OF THE INVENTION

Embodiments are directed towards an ultrasound instrument and a method of operating an ultrasound instrument and, in particular towards ultrasound instruments that can, in use, mediate delivery of chemotherapy drugs to cells of diseased tissue such as a cancer tumor, and to methods of operating such instruments.

### BACKGROUND OF THE INVENTION

### Therapeutic Agent

Cancer chemotherapy drugs are generally administered through the blood. The drugs cross the capillary wall and transports through the tumor interstitium (external-capillary space between cells) to the cancer cells. However, drugs with the small molecules also cross the capillary wall in normal tissue, producing some damage also to normal tissue.

Due to the aggressive growth of malignant tumors, the capillaries of the tumor have imperfect walls. Many research groups are working on packaging the drug into nano-sized particles (diam ˜100 nm) that crosses the imperfect capillary wall of tumors, but not the capillary wall of normal tissue, hence protecting the normal tissue against the drug. The drug is either encapsulated in the nanoparticles by a shell, or dispersed throughout the nanoparticle. There are also in development drugs with large molecules, where the dimension of each molecule is >10 nm, which have similar properties for crossing the capillary wall in tumor and normal tissue, as nanoparticles. Examples of large molecular drugs are found in modern gene therapy, both for treatment of cancer and other diseases, such as malfunction of muscle cells in heart failure, and neuro-degenerative diseases.

There is further in development micro-bubbles (diam ˜3 μm) with a shell containing drugs either in molecular form or a shell formed of nanoparticles containing drugs. These micro-bubbles scatter ultrasound very strongly nonlinearly, thereby improving separation of signal scattered by the micro-bubbles from signal scattered by tissue. Several known methods to achieve such separation are known. The sensitivity of such methods can be such that single micro-bubbles in tumor capillaries can be detected. Controlled ultrasound breakage of the micro-bubbles can be employed to release the drugs either in molecular form or as several thousands of drug-containing nanoparticles that can cross the capillary wall into the interstitium of tumor tissue.

There is further in development drug delivery systems, where the drugs are dispersed in micro-droplets (diam ˜3 μm). Ultrasound heating (UH) is used to evaporate the droplets, releasing the drug in the form of molecules or nano-particles that can cross the capillary wall. Nano-droplets (diam ˜100 nm) containing material where ultrasound can be used to stimulate phase change from fluid or solid to gas, are designed to leak out into the tissue interstitium. Ultrasound stimulated phase change then generate gas micro-bubbles (diam ˜3 μm) in the interstitium, that can be detected with ultrasound as a contrast agent, and also used to generate pores (called transient sonoportation) in the cancer cell membranes for improved transport of large molecules or particles into the cancer calls.

In the following the term therapeutic agent is used for drugs in molecular form of any size, and also drugs encapsulated or dispersed in nanoparticles, and drugs used in combination with other means, such as radiation therapy.

### Inhibitions to Transport of Drugs to Diseased Cells

The wall imperfection of tumor capillaries produces excessive leakage of fluid from the capillaries into the tumor interstitium, and combined with an imperfect lymphatic drainage in the tumor, the tumor interstitial fluid has increased pressure compared to normal tissue. This gives a very low pressure gradient from the tumor capillaries and throughout the tumor interstitial fluid, lowering pressure gradient forced convection of therapeutic agent into and within the interstitium. The driving force for transport of therapeutic agent is in this situation mainly given by diffusion, which is a slow process driven by the spatial concentration gradient of the agent. Biological membranes, such as the wall of cells and the blood brain barrier generally inhibit the transport of drugs and genes in the form of large molecules or nanoparticles, for various kinds of therapy.

We shall also use the term diseased tissue to describe tissues where the treatment according to this invention are useful, for example cancer tumors, malfunction of muscle cells in heart failure, and neurodegenerative diseases.

### SUMMARY OF THE INVENTION

An overview of embodiments of the invention is presented. The overview is meant for illustration purposes only, and by no means represents limitations of the invention, which in its broadest aspect is defined by the claims appended hereto.

Instruments and associated operating methods are disclosed that, based on input that may relate to location and type of diseased tissue, type of surrounding tissue, and type of treatment, determine appropriate variation of one or more of ultrasound beam frequency, intensity, focusing, mechanical index or scanning pattern or rate, embodiments to enable the use of ultrasound, when applied to a patient having had a therapeutic agent administered, for one or more of three separate functions in relation to improved delivery of therapeutic agent to cancer and other diseased cells.

1. According to an aspect of the invention there is provided an instrument suitable for ultrasound mediated transport of a therapeutic agent in a region of diseased tissue. This instrument comprises a transmitter for transmitting at least one ultrasound radiation force beam, hereinafter referred to as URF beam, for insonifying at least a region of tissue. At least one of a scanning rate for scanning the beam across a region of tissue, a transmit focus, a transmit aperture, and a transmit frequency of the URF beam can be selected by the instrument. The instrument further comprises a processor that is configured to calculate, based on an indication of a tissue type or of tissue types to be insonified and on an indication of a depth range within which ultrasound intensity and extinction of intensity is to be increased, at least one of the following transmit parameters for said at least one URF beam:
i) a number of URF beam transmit foci to be used,
ii) respective depths for one or more URF beam transmit foci,
iii) a URF beam transmit aperture for the URF beam transmit focus or respective URF beam transmit apertures for each of more than one URF beam transmit foci,
iv) a URF beam transmit frequency for a URF beam transmit focus or respective URF beam transmit frequencies for each of more than one URF beam transmit foci,
v) a URF beam transmit pressure for a URF beam transmit focus or respective a URF beam transmit pressures for each of more than one URF beam transmit focus,
vi) a URF beam scanning region, and
vii) a URF beam scanning rate.

The instrument further comprises set up means for setting up the transmitter with the selected transmit parameter or transmit parameters for the URF beam.

The transmitter and the scanning means may comprise means for insonifying at least one region of tissue with URF beams from multiple directions at least one of sequentially and simultaneously.

The means to calculate at least one transmit parameter is configured to base its calculations on stored data encoding the depth dependency of at least one of the transmit parameters. This data can be stored in the processor or elsewhere in the instrument. The calculations may, for example, be based on simulations of wave propagation in tissue, for example as described in the Appendix, or on data obtained from such simulations. Alternatively or additionally, the data relied upon in the calculations may be based on experiments determining the depth dependent of one or more of the at least one of the transmit parameters. The instrument may further be configured to alter and adapt the data relied upon in the calculations through learning from the results of treatments to further improve its ability to determine the transmit parameters. The instrument may, for example, store parameters used for insonifying patients and further provide means for receiving an input relating to the efficacy of the treatment, once such efficacy can be determined by a medical practitioner. The instrument may then, based on such newly received data, alter and adapt the way it proposes transmit parameters. Computer implemented ways of learning from such data are known and can for example include neural networks.

Selecting a frequency for substantial maximization (i.e. under given practical constraints as limited knowledge of tissue parameters, available transmit power, and ultrasound transducer bandwidth) of ultrasound intensity and extinction of intensity (sum of scattering and thermal absorption) from the incident beam inside the diseased tissue, one substantially maximizes the ultrasound radiation force (URF) onto a small volume element that can be used to increase the convection of therapeutic agent from the capillaries of the diseased tissue deep into the interstitium of the diseased tissue, and across biological membranes such as the capillary wall, the cell wall, and the blood brain barrier. Simulation of the frequency dependency of the URF at a focal depth within the diseased tissue, for example according to Eq. (A11) and the methods described in the Appendix and implemented in SW in the processor and exemplified in FIG. 6a, is useful for finding a frequency for substantial maximum of the URF. Reference to a substantial maximum is made herein, as the actual acoustic parameters of the diseased and surrounding tissue are generally not accurately known. In practical situations a further limiting factor is the bandwidths of available ultrasound transducer arrays and simulations may need to take such available bandwidth into account or an ultrasound apparatus arranged to select an US frequency for insonification may do so based on simulation results as restricted by available bandwidth. Typical frequencies for achieving a high URF are in the range of ˜3-12 MHz for depths in the range of ˜20-120 mm.

2. A lower frequency ultrasound beam that increases the mechanical index, hereinafter referred to as MI, of the ultrasound, is useful for breakage of particles with drugs encapsulated or dispersed, to release the active drug molecules within the diseased tissue, and also to stimulate transport of particles across biological membranes such as the cell wall or the blood brain barrier. High MI frequencies are less than ⅕ of the URF frequency, typically ˜0.2-0.6 MHz, and a MI of more than 0.5, typically >2. The higher frequency for ultrasound radiation force (URF) or ultrasound heating (UH) under point 1 and 3 produces low MI with low particle breakage (See Eq. (6) and FIG. 2).

In an embodiment the transmitter is suitable for transmitting high MI beams either sequentially or simultaneously with high URF beams.

3. Selecting the frequency for substantial maximal thermal absorption of the ultrasound beam in a region, one can use the ultrasound for therapeutic heating of the tissue, for example to increase blood flow to the diseased tissue, increasing the flow of therapeutic agent and oxygen to diseased tissue, direct thermal destruction of diseased tissue, and also to manipulate thermally sensitive particles. Simulation of the frequency dependency of the UH at a focal depth within the diseased tissue, for example according to Eq. (A13) and the methods described in the Appendix and implemented in SW in the processor and exemplified in FIG. 6b, is useful for finding a frequency for substantial maximum of the UH. Reference to a substantial maximum is again being made, as the actual acoustic parameters of the diseased and surrounding tissue are generally not accurately known. A further limitation in practical situations is again the bandwidths of available ultrasound transducer arrays, which may also be accounted for when basing a selection on simulation results. Useful ultrasound frequencies for maximal heating are ˜⅕-⅓ of the frequencies for high/maximal URF.

In an embodiment the transmitter is configured to transmit, simultaneously or sequentially with said URF beam, an ultrasound heating, hereinafter referred to as UH, beam for therapeutic heating of tissue with at least one UH beam, where at least one of a scanning rate for scanning the beam across a region of tissue, a transmit focus, a transmit aperture and a transmit frequency of said UH beam can be selected. In the embodiment the processor is configured to calculate, based on an indication of a depth range within which the tissue is to be heated, at least one of the following transmit parameters for said UH beam:
i) a number of UH beam transmit foci to be used,
ii) respective depths for one or more UH beam transmit foci,
iii) a UH beam transmit aperture for the UH beam transmit focus or respective UH beam transmit apertures for each of more than one UH beam transmit foci,
iv) a UH beam transmit frequency for a UH beam transmit focus or respective UH beam transmit frequencies for more than one UH beam transmit foci,
v) UH beam transmit pressures for each transmit focus, and
vi) a UH beam scanning region,
vii) a UH beam scanning rate.

The instrument of the embodiment further comprises a set up means for setting up the transmitter with the calculated transmit parameter or transmit parameters for said UH beam.

The processor may further be configured to calculate the transmit parameters of the at least one URF beam so that production of therapeutic (heating) hyperthermia by the URF beam is substantially avoided. This can be achieved through simulation of the wave propagation in the tissue, for example according to the methods described in the Appendix and implemented in SW in the processor, where Eq. (A13) gives the heat energy delivered at a given depth in the tissue. To avoid therapeutic hyperthermia, the transmit amplitude is limited so that the delivered heat is below a limit established from experiments.

In one embodiment function 1 above is used sequentially with one or both of functions 2 and 3 discussed above. These functions may be used in a freely selectable sequence. Alternatively or additionally function 1 can be used simultaneously with function 2 and/or function 3, for example for improving the delivery of therapeutic agent to diseased cells. The processor of any of the aforementioned instruments may further be configured to support the instrument operator in planning a sequence of treatment scans. The treatment beam scans may be composed of one, two or all three of an URF beam scan, an high MI beam scan, and an UH beam scan. The different scans can be set up in a freely selectable order to be performed in a sequence, simultaneously or in a combination of sequential and simultaneous beam application. The set up means is configured to set up the instrument to apply the planned sequence of treatment scans.

The instrument may further comprise means for generating an image of the diseased tissue and means for defining the region of diseased tissue in the image, the means for calculating the transmit parameters can in this case be configured to use the defined location of the diseased tissue as input. Direction and foci of the ultrasound beams can be guided using acquired images. The instrument can further be configured to increase MI to, based on acquired cavitation images, increase cavitation and/or to decrease MI to decrease cavitation and/or to adjust MI to obtain a cavitation level close to a selected level and/or to, based on temperature images of insonified tissue, select UH beam transmit parameters to obtain a tissue temperature close to or not exceeding a desired/predetermined temperature. Several imaging modalities, such as ultrasound imaging, photo-acoustic imaging, magnetic resonance imaging, X-ray imaging, nuclear imaging, and optical imaging, can be used. The embodiments also make use of contrast agents adapted to the imaging modality, for improved information extraction from the images. Imaging contrast agents may be used. The images acquired can be 3D images to allow full quantification/outlining of tissue to be insonified.

According to another aspect of the present invention there is provided a method for ultrasound mediated transport of a therapeutic agent in biological tissue. The method comprises calculating for a given depth range to be insonified, at least one of the following transmit parameters for use with at least one ultrasound radiation force beam, hereinafter referred to as URF beam:
i) number of URF beam transmit foci to be used,
ii) respective depths for one or more URF beam transmit foci,
iii) a URF beam transmit aperture for the URF beam transmit focus or respective URF beam transmit apertures for each of more than one URF beam transmit foci,
iv) a URF beam transmit frequency for a URF beam transmit focus or respective URF beam transmit frequencies for each of more than one URF beam transmit foci,
v) a URF beam transmit pressure for a URF beam transmit focus or respective a URF beam transmit pressures for each of more than one URF beam transmit focus,
vi) a URF beam scanning region, and
vii) a URF beam scanning rate.

The method further comprises transmitting the at least one URF beam using said calculated transmit parameter.

The ultrasound beam can be transmitted from a wide aperture with strong focusing to achieve a high radiation force in the beam focal region. The focal region is scanned across said diseased tissue.

Multiple focal regions may be applied sequentially along at least one beam direction to cover the diseased tissue with focal regions.

The at least one URF beam may be scanned so that a region of tissue is insonified with URF beams in multiple directions at least one of sequentially and simultaneously.

The transmit parameters of the at least one URF beam can be selected so that production of therapeutic hyperthermia by the URF beam is substantially avoided and/or so that the URF beam also generates therapeutic hyperthermia of a region of the diseased tissue. Therapeutic hyperthermia, if generated, can be used to heat thermally sensitive particles to one or both of release therapeutic agent and generate gas bubbles in the diseased tissue.

A high MI beam at a frequency lower than ˜⅕ the frequency of the URF beam, typically of the order of ˜0.2-0.6 MHz, can further be submitted one of sequentially and simultaneously with said URF beam. The at least one high MI beam can be scanned across said region of the diseased tissue with selectable MI beam scanning region and MI beam scanning rate.

### BRIEF DESCRIPTION OF THE DRAWINGS

FIG. 1 Illustrates an example of 3D ultrasound imaging of a diseased tissue, for example a tumor, to guide transmission of treatment ultrasound beams for mediation of delivery of drugs to diseased cells.

FIG. 2 Shows the percentage release of drugs from encapsulating nanoparticles through insonification with ultrasound at 0.3 and 1 MHz, as a function of mechanical index, MI, of the ultrasound

FIG. 3 Shows an example block diagram of an instrument according to an embodiment.

FIGS. 4A and 4B Show examples of arrangements of transducer arrays for scanning the ultrasound beams from different directions.

FIG. 5 Illustrates a flow chart of typical use of an instrument for imaging, planning of ultrasound treatment scanning, and the carry trough of ultrasound mediated treatment of diseased tissue.

FIGS. 6A and 6B are graphs showing typical examples of variation with frequency of ultrasound radiation force at the focal depth of 22 mm, and ultrasound heating of tissue at the focal depth of 22 mm, obtained by simulation of wave propagation in tissue with nonlinear elasticity.

FIGS. 7A and 7B are graphs showing typical examples of variation with depth of ultrasound radiation force at 11 MHz, and ultrasound heating of tissue at 4 MHz, obtained by simulation of wave propagation in tissue with nonlinear elasticity.

### DETAILED DESCRIPTION OF EMBODIMENTS OF THE INVENTION

Example embodiments of methods and instrumentation according to the invention are presented in the following. This presentation is meant for illustration purposes only, and by no means represents limitations of the invention, which in its broadest aspect is defined by the claims appended hereto.

### Ultrasound Radiation Force (URF) for Transport of Therapeutic Agent

An ultrasound beam propagating in a medium that one or both of scatters and absorbs the ultrasound wave, exerts a radiation force onto the scatterers and the medium. The radiation force is in the direction of the incident wave and proportional to the extinction of intensity of the incident wave from the volume due to scattering and absorption of wave intensity. Embodiments provide means for adjusting an ultrasound aperture, focus, and frequency so that, in use, it can apply an URF to increase the transport of therapeutic agent from the capillaries of the diseased tissue, deep into the tissue interstitium, and also through biological membranes like the capillary wall, the cell wall, and the blood brain barrier.

Ultrasound scattering and absorption are produced by particles and molecules in the volume, and one can consider the URF as acting on the individual particles or molecules. However, when the particles or molecules are much smaller than the ultrasound wavelength and the dimension scale that is appropriate to analyze transport dynamics in the system, it is useful to consider the ultrasound medium as a continuum. A small volume ΔV of the medium then produces an extinction of the incident ultrasound intensity due to a large variety of molecules and particles in the volume that both scatters and absorbs the ultrasound intensity. As the therapeutic agent generally is dissolved in a fluid, the URF on a small volume has the similar effect as a pressure gradient, and produces convection of the fluid and the therapeutic agent. The combined scattering and absorption of the incident intensity defines the extinction cross section σₑ(ω) with depth as

dI(ω)=−σₑ(ω)I(ω)ds

σₑ(ω)=σₐ(ω)+σₛ(ω) (1)

where ω=2πf is the angular frequency components of the incident wave, I(ω) is the frequency distribution of ultrasound intensity incident onto the volume element, ds is an infinitesimal propagation distance along the beam direction, σₛ(ω) is the scattering cross section and σₐ(ω) is the absorption cross section in the volume element. The intensity is proportional to the square of the ultrasound pressure. We can express the URF, ΔF, on the volume element in the direction of the incident wave as

$$Δ F ( ω ) = \frac{{σ}_{e} ( ω ) I ( ω )}{c} Δ V \qquad ( 2 )$$

As described, the URF has similar function as a pressure gradient in that it forces convection of particles and fluids containing therapeutic agent. URF can hence be used for improving transport of therapeutic agent from the capillaries of the diseased tissue and deep into the tissue interstitium, and also across biological membranes and into diseased cells.

Particles in a fluid flow have a tendency to move towards the region of lowest gradient in the fluid velocity. Nano-particles or large drug molecules therefore have a tendency to move towards the center region of the capillaries, where the gradient of the blood velocity profile is small. An URF transverse to the capillary direction will hence also increase the transport of therapeutic agent from the blood across the capillary wall into the interstitium, in addition to increased transport of the agent deeper into the interstitium. The capillaries of diseased tissue, especially tumor tissue, are usually chaotically arranged with close to even direction distribution in all directions. For an even distribution of capillary direction in all directions and radiating the ultrasound beam in a single direction, the URF will be efficient for improving transport of therapeutic agent out of the capillary for ˜60% of the capillaries. It can however be useful to radiate the ultrasound beam in several directions into the diseased tissue to increase this percentage. To allow increasing the transport of therapeutic agent from the blood across the capillary wall into the interstitium, when an instrument according to an embodiment is used on a patient, the embodiments also allow adjustment of an ultrasound instrument and probe to generate ultrasound beams with different directions, either sequentially or simultaneously.

### Optimizing Frequency and Focus for URF

The therapeutic agent particles and molecules are small compared to practical ultrasound beam widths, and are found in a fluid with fairly low viscosity. The lateral velocity coupling in this fluid across the beam width, is low. From Eq. (2) we see that the URF is proportional to the incident intensity of the ultrasound beam onto the volume element ΔV. Hence, to maximize the URF in a region of interest within the diseased tissue, one should maximize the ultrasound intensity, which is achieved by focusing the URF beam in the region and select an ultrasound frequency which substantially maximizes the intensity extinction in the beam focus.

The on-axis intensity I of the beam attenuates due to the intensity extinction for linear wave propagation as

I(z,ω)=I₀(z,ω)exp{−∫₀^{z}dsσₑ(ω,s)} (3)

where I₀(z,ω) is the on-axis intensity of the ultrasound beam at depth z without scattering and absorption in the material. The exponential term represents beam attenuation due to extinction of intensity with depth due, and as σₑ>0 and increases with frequency, this term implies that the frequency for maximal intensity drops with increasing depth.

The intensity I₀(z_{f},ω) without extinction in the focal point at depth z_{f} is

$$\begin{matrix}{I}_{0} ( {z}_{f} , ω ) = {I}_{0} ( 0 ) {( \frac{{A}_{i}}{λ {z}_{f}} )}^{2} \\ = {I}_{0} ( 0 ) \frac{{A}_{i}}{{λ}^{2} {FN}_{a } {FN}_{e}}\end{matrix} \qquad ( 4 )$$

without I₀(0) is the average intensity across the transmit aperture surface, Aₜ is the area of the transmit aperture, and λ is the acoustic wave length. For a rectangular transmit aperture, which often is used, we have Aₜ=DₐDₑ, where Dₐ is the azimuth width of the aperture and Dₑ is the elevation width of the aperture. FNₐ=z_{f}/Dₐ and FNₑ=z_{f}/Dₑ are the azimuth and elevation f-numbers, respectively. We notice that without scattering and absorption, i.e. σₑ=0, and within the regime of linear acoustics, the focal intensity I₀(z_{f},ω) is ˜Aₜ² and ˜1/FNₐ and 1/FNₑ. Due to diffraction we have I₀(z_{f},ω)˜λ⁻²˜ω² in the fully homogeneous material. In the practical heterogeneous materials we have aberrations of the acoustic wave front that reduces this diffraction increase of focal intensity with frequency. In the Appendix it is shown how one can correct for the decreasing effect of wave front aberrations on the focal intensity.

The radiation force of Eq. (2) in within the realm of linear acoustics given as

$$Δ F ( z , ω ) = \frac{Δ V}{c} {I}_{0} ( z , ω ) {σ}_{e} ( ω , z ) exp \{ - {\int }_{0}^{z} d s {σ}_{e} ( ω , s ) \} \qquad ( 5 )$$

Within the realm of linear acoustics, maximization of URF implies maximization of this equation, and a 1^{st} approach to maximum of the URF in a region of the diseased tissue, is within the realm of linear acoustics found by placing the beam focus in the middle of the region and finding the frequency that maximizes Eq. (5) for the focal depth z_{f}. We notice from the mid term of Eq. (4) that the focal URF is proportional to Aₜ², and inversely proportional to z_{f}². The last term of Eq. (4) states that the focal URF is proportional to Aₜ, and inversely proportional to FNₐ and FNₑ. These are hence the aperture parameters we can manipulate with to maximize the URF in a region.

For the absorption cross section one finds experimentally σₐ˜ω^{b} where typically b˜1-1.5 for medical ultrasound frequencies in soft tissues, where b≈1 in most cases with an absorption ˜0.5 dB/cmMHz. The scattering cross section depends on the particle size distribution in relation to the ultrasound wavelength. An adequate approximation for medical ultrasound frequencies and particle sizes found in soft tissue is a scattering cross section σₛ˜ω^{a} where a<4, where the scattering cross section generally is ˜0.1 of the absorption cross section.

In the practical situation, the forward wave oscillation is nonlinearly distorted due to nonlinear tissue elasticity, producing nonlinear wave propagation. One does not have exact formulas for the wave intensity with nonlinear wave propagation, and computer simulations are required to determine the beam intensity, for example as described in the Appendix. The simulation parameters are determined by the tumor tissue type, for example obtained from experiments and experience and stored in the instrument processor memory. The oscillation distortion produces higher harmonic components of the transmitted frequency components coin the tissue. As σₑ(ω) increases with frequency, the nonlinear distortion increases the URF above that given by linear analysis of wave propagation, increasing the harmonic components and the intensity extinction especially within a high intensity focal region. To get a high URF within the diseased tissue, the instrument according to embodiments can use a wide transmit aperture, with strong focusing, i.e. low f-numbers, of the intensity within the diseased tissue to be treated, to obtain high nonlinear distortion of the wave oscillation in the focal region giving high intensity extinction and a high URF in the focal region.

To select an ultrasound radiation aperture and frequency that produces a high URF in the focal region, a simulation of the ultrasound wave propagation with nonlinear tissue elasticity, and with selected tissue parameters is used, for example as presented in the Appendix. Such simulations will deviate from the real situation in tissue, because the material parameters prevalent in tissue that is to be irradiated are not known exactly, and as material heterogeneity produces aberrations of the focused wave front (wave front aberrations) which defocuses the beam so that for high frequencies the frequency variation of I₀ drops from the ˜ω² variation. However, simulations of this nature can provide an interesting accurate estimate of the frequency for substantial maximum URF generated in tissue. As shown in the Appendix, one can also correct for the effect of wave front aberrations on the focal intensity.

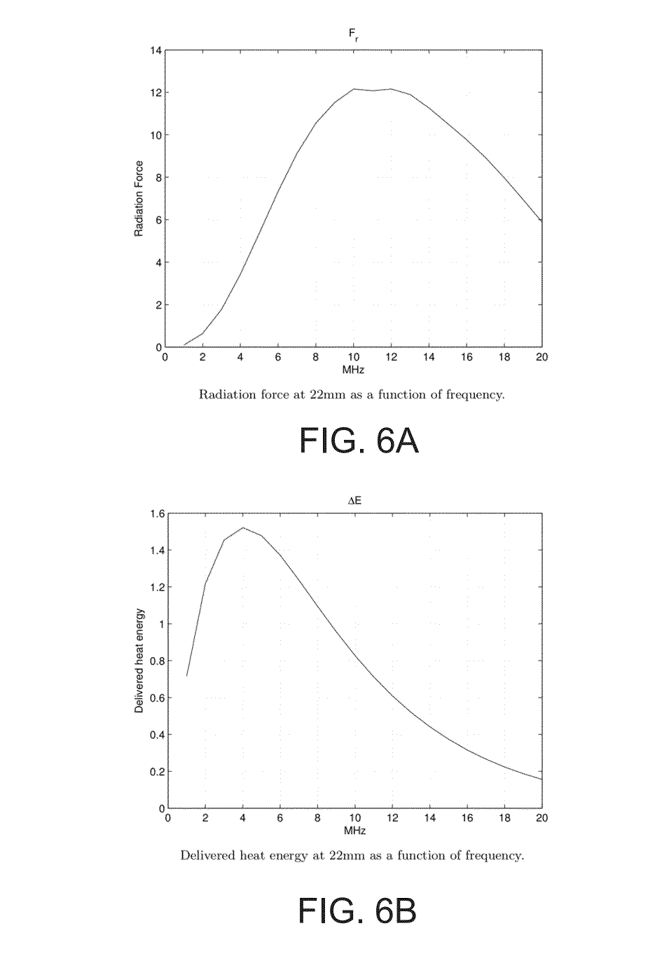

FIG. 6a shows a simulation of the focal URF as a function of the center frequency of the transmitted pulse. The simulation methods used are described in the Appendix, with r=rₜ in Eq. (A11) giving the URF in the transmit beam focus. The transmit pressure on the array surface is 1 MPa, the focal depth is 22 mm, the azimuth aperture is 14.4 mm giving FNₐ≈1.5, and the elevation aperture is 4.3 mm giving FNₑ≈5. The parameters of the extinction cross section is as given in relation to Eq. (A8). For these parameters we get a maximum URF at 11 MHz, which is a typical situation for the given depth of 22 mm. The exponential function (extinction) in Eq. (5) will reduce the frequency for maximum URF with increasing depth. For liver and kidney the frequency for maximal URF is ˜3-5 MHz, depending on depth. With constant transmit pressure P₀, the total transmitted power is ˜Aₜ. Increasing Aₜ reduces the focal width, so that the focal intensity, and thereby the URF is ˜Aₜ² and ˜ω² for linear wave propagation, as shown in Eq. (4).

The frequency bandwidth of efficient URF in FIG. 6a is quite broad, providing more than 75% of the maximum URF in the band of 8-14 MHz. As wave front aberrations will reduce the frequency of the URF maximum, it makes sense to use an ultrasound URF frequency that is on the low side of the maximum in the simulations. The broad bandwidth indicates that such simulations can be used for selecting an ultrasound frequency that substantially maximizes the URF in the focal region inside the diseased tissue, even though the material parameters and degree of wave front aberrations are not exactly known when the simulation is conducted. In instruments according to the invention, the invention devices store typical acoustic parameters of different types of normal and diseased tissues. For planning the URF therapy scanning of diseased tissue of a given type in a given organ, the stored parameters for the given organ and diseased tissue are used in simulations to select aperture and frequency for substantially maximizing the URF for the therapy scanning for the given case. We also note that the radiation force increases with the square of the transmitted pressure amplitude.

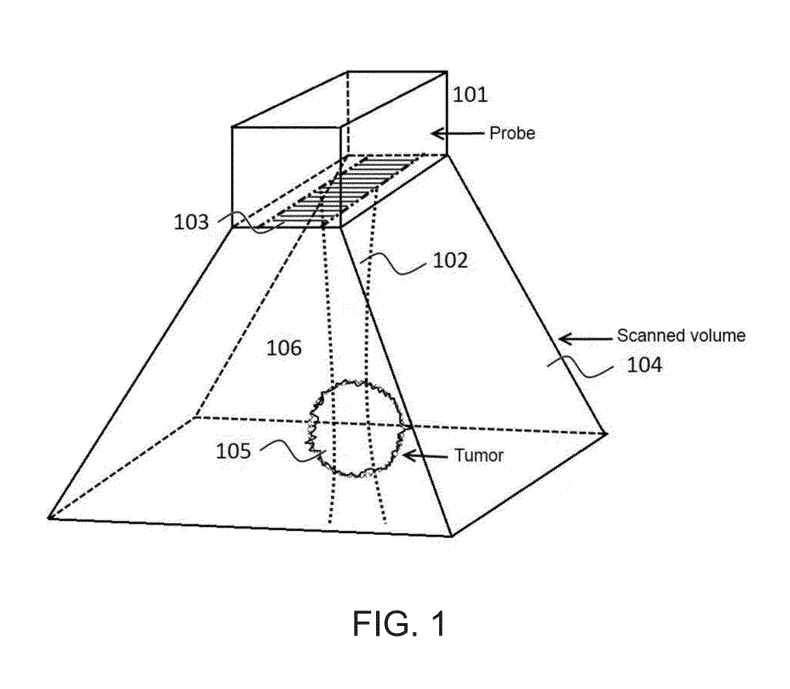

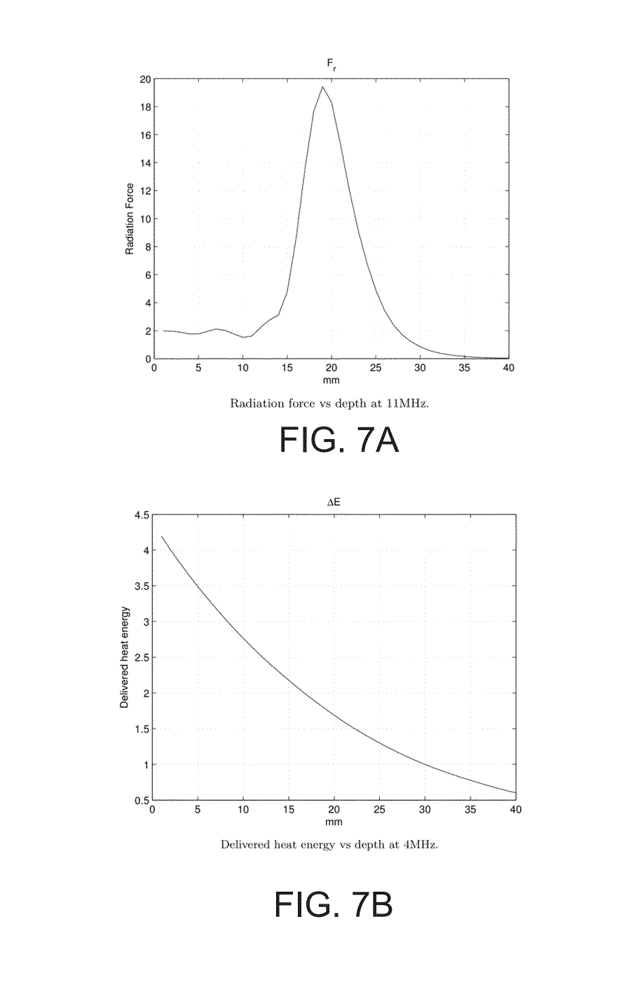

FIG. 7a shows an example simulation of the variation with depth of the URF for the transmitted ultrasound center frequency of 11 MHz. The same simulation parameters as in FIG. 6 are used. To maximize the URF at a selected point, the focus should be at this point. There is hence a focal region where intensity is high, according to Eqs. (5, A11), and the 3-dB length of the focal region for the URF magnitude is ˜5 mm in this particular situation. The length of the 3-dB focal region is ˜7λ FNₐ FNₑ. To allow covering the diseased tissue with URF, the instrument according to embodiments are configured to, in use, to scan the focal depth and the beam direction of the focused ultrasound beam in a 3D manner across a region of diseased tissue to be treated. When the depth extension of the diseased tissue along the beam is adequately low, a beam could be scanned with a single focus for each beam direction, but for treatment regions with a large extension along the beam, it is advantageous to use multiple transmit foci along at least some beam directions. In these situations, instruments according to embodiments also can be set up to use low diffraction ultrasound beams such as Bessel beams and beams generated with a conical focusing of the transmit aperture, both of which have specially long focal regions, albeit with reduced maximal intensity. Scanning of the focal depth are obtained by setting transmit delays for the array transmit channels according to methods known to anyone skilled in the art. Scanning the direction of the ultrasound beam can be done by mechanical movement of the array in combination with electronic beam steering, where for 2D matrix arrays the direction scanning is done fully electronically, as discussed in relation to FIG. 1 below.

### Imaging for Guidance of URF Beam and Focal Scanning

Instruments according to the invention use a 3D image of the diseased tissue as a basis input to guide steering of the scanning of the URF beam direction and foci. Such an image can for example be obtained by 3D scanning of an ultrasound image beam, as illustrated in FIG. 1. This image shows as 101 an ultrasound probe that is able to steer an ultrasound transmit/receive beam 102 from a linear array 103 within a 3D volume 104 to provide a 3D image of the diseased tissue 105, for example a tumor, and surrounding tissue 106. Ultrasound imaging with dual band pulse complexes as described in U.S. Pat. No. 8,038,616 and U.S. patent application Ser. No. 12/500,518 (2010/0036244) is then useful for improved differentiation between diseased and surrounding tissue, especially by using ultrasound contrast agent for tumors with increased density of capillaries due to neo-angiogenesis.

Other imaging modalities, such as photo-acoustic imaging, magnetic resonance (MR) imaging, X-ray computer tomography imaging, or nuclear imaging including positron emission tomography (PET) imaging, can be used for defining the diseased tissue to guide the scanning of the URF beam and focus in the same manner. For shallow regions of diseased tissue it is also useful to use optical imaging of the diseased tissue such as low coherence tomography optical imaging, for example for skin or eye imaging, or diseased tissues that can be imaged through an endoscopic probe, such as tissues in the intestines, the prostate, the urinary system, in gynecology, or tissues that are accessible during surgery.

For enhanced definition of the diseased tissue, it can also be advantageous to use contrast agents adapted to the imaging modality. Contrast agents that stay in the blood vessels for adequately long time, can be used to show increased angio-geneses, typical of aggressively growing tumors and some atherosclerotic tissue. Contrast agents adapted to leak into the interstitium of tumors, but not into interstitium of normal tissue, are also useful to define the tumors in the image. One can similarly use contrast agents that are targeted to the biology of diseased tissue to accumulate in the diseased tissue.

Based on the image of the diseased and surrounding tissue, the boundaries of the diseased tissue are extracted, either automatically through image analysis, or by manual interaction by an operator, or a combination of both. The detected boundaries of the diseased tissue in the image can then be used for defining the scanning of the URF beam direction and focus, and also other therapy beams, such as high MI beams and high UH beams discussed below. To achieve this, embodiments of the invention have a well defined geometric relationship between the image pixel coordinates and coordinates of the therapy beams and foci.

### High MI Beams for Particle Breakage and Stimulation of Transport Across Membranes

For drugs that are encapsulated or dispersed in particles, one can obtain ultrasound mediated breakage of particles through insonification with high mechanical index ultrasound. The mechanical index (MI) of the ultrasound is defined as

$$MI = \frac{{P}_{neg}}{\sqrt{f}} \qquad ( 6 )$$

where P_{neg} is the negative amplitude of the ultrasound pressure oscillation, and f is the ultrasound frequency. We hence see that lowering the ultrasound frequency increases the MI for constant negative pressure. The ultrasound intensity is determined by the pressure squared. The MI hence is increased by a lowering of the frequency for a given acoustic intensity and URF. The extinction and absorption also reduces with frequency, so that reducing the frequency reduces the URF of the ultrasound beam, and also UH as discussed below, while allowing an increase in the MI. Frequencies for high MI beams are lower than ˜⅕ of that used for URF, typical of the order of 0.2-0.6 MHz.

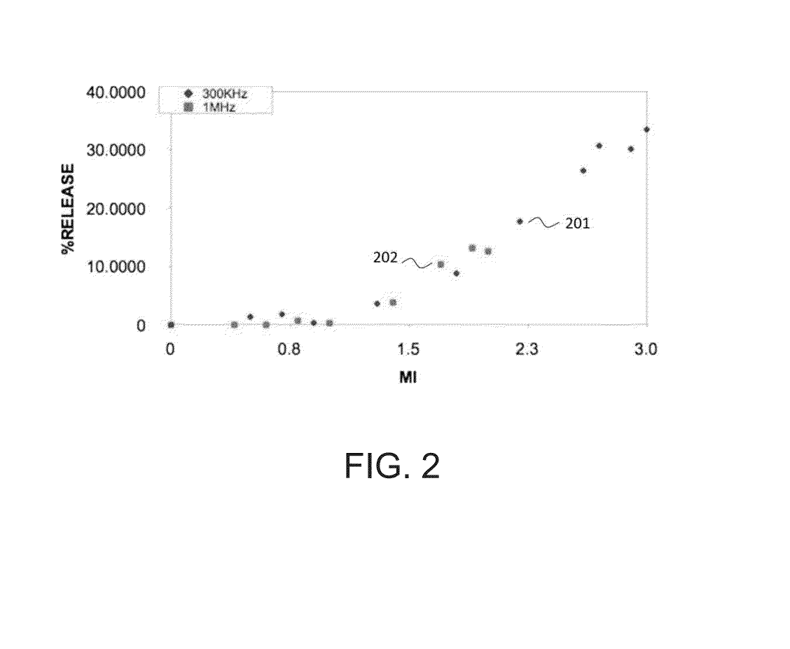

In FIG. 2 is shown the effect on ultrasound breakage of nano-particles by an incident ultrasound wave. 201 shows the release of drug as a function of MI for 0.3 MHz ultrasound, while 202 shows the release of drug as a function of MI for 1 MHz ultrasound. We see that the release of drug is mainly determined by the MI and not by the ultrasound frequency per se. For MI<1.2 there is practically no release of drug for both frequencies, while the percentage release increases close to proportional to the MI above this limit. Further studies show that the drug release is an exponential increase with time as

R(t,MI)=R₀(MI)(1−e^{-t/T}^{ᵣ}^{(MI)}) (7)

where R(t,MI) is the percentage release as a function of ultrasound insonification time t and incident mechanical index MI, Tᵣ(MI) is the time constant of the release, and R₀(MI) is the maximal release shown in FIG. 2 for long insonification times. For efficient breakage of nanoparticles MI>2 are useful. Breakage of micro-bubbles where the therapeutic agent is loaded in the shell can also be done with ultrasound, where the limit for breakage is also determined by the MI, albeit at a lower MI limit for micro bubbles, typically a MI˜0.5, than for nanoparticles.

Low frequency (<3 MHz) high mechanical index ultrasound has also experimentally been shown to increase transport of larger molecules and particles across biological membranes, such as the cell wall and also the blood brain barrier. The mechanism for breakage of particles and stimulated transport through biological membranes has been hypothesized to originate from cavitation of small gas bubbles in the tissue, producing both breakage of particles and transient sonoporation. The instrument can therefore favorably be operated together with micro-bubbles artificially introduced to the capillaries to stimulate the opening of the blood brain barrier. It has also been shown that remodeling of myocardial tissue in the border zone of an infarction is stimulated by low frequency ultrasound insonification of the border zone containing microbubbles in the capillaries. Cavitation can according to known methods be monitored by ultrasound imaging or magnetic resonance imaging. There are also indications that a main mechanism for transport across cell wall membranes is stimulation of endocytosis of particles into the cells by the high MI beam.

### Ultrasound Heating (UH)

Ultrasound can also be used to heat the tissue (UH), as for example done in HIFU (High Intensity Focused Ultrasound) treatment used to directly destroy the diseased tissue with high temperature (˜60 deg C.). Lower increased temperatures can be used to increase blood flow for increased inflow of therapeutic agent to the diseased tissue, and also increased inflow of oxygen to the diseased tissue that increases the efficacy of some therapeutic agents. Ultrasound heating has also been used for phase transition of material components in micro- and nano-particles from solid/fluid state to gas state to release drugs, generate gas bubbles that can be imaged as a contrast agent with ultrasound or be used to stimulate transport of therapeutic agent through biological membranes.

The heat energy delivered to the tissue per unit time is given by the absorption of the ultrasound power in the beam, and the total delivered heat energy to a region is in the regime of linear wave propagation proportional to

˜TₕW₀σₐ(ω,z)exp{−∫₀^{z}dsσₑ(ω,s)} (8)

where Tₕ is the heating time that the beam is adequately stationary in the region, and W₀ is the power in the beam neglecting scattering and absorption.

For UH of the tissue it is the total power in the beam that enters, rather than the beam intensity as for the URF in Eq. (5), because the tissue region covered by the beam is cooled by neighboring tissue due to blood flow and thermal diffusion. UH of tissue therefore has much stronger spatial coupling to neighboring tissue than the URF in a fluid has. As stated above, we utilize the URF acting on a fluid with limited viscosity, and its effect on transport of therapeutic agent hence has a much weaker spatial coupling than UH of the tissue. It is hence the local, on axis beam intensity I₀ that enters for the URF in Eq. (5), while it is the total power W₀ in the beam that enters into Eq. (8) for UH. We note that I₀ increases ˜ω² due to diffraction within the linear propagation regime, while W₀ is independent of ω, and this gives a 1^{st} effect that makes the frequency for maximal URF higher than for UH. UH is therefore also less sensitive to defocusing of the ultrasound beam due to tissue heterogeneities and wave front aberrations caused by such heterogeneities. Further, the UH is proportional to the thermal absorption cross-section, σₐ, while the URF is proportional to the total extinction cross section, σₑ=σₛ+σₐ. As described above, σₛ˜ω^{a} where a˜<4 depends on particle size in relation to the wavelength, while σₐ˜ω^{b} where one experimentally finds b˜1-1.3 for soft tissues. This gives a 2^{nd} effect that gives a higher frequency for maximal URF compared to for UH.

Nonlinear wave propagation introduces harmonic components of the incident frequencies, and hence increases the absorption and the UH. To maximize UH, numerical simulations of the nonlinear wave propagation are used, for example according to the methods described in the Appendix, where Eq. (A13) gives the total heat energy delivered by the UH beam at a given depth. An example is shown in FIG. 6b which shows the heat delivered to the tissue at a focal depth of 22 mm as a function of center frequency of the transmitted burst. The same tissue parameters and transmit aperture is used as in FIG. 6a. FIG. 6b shows a maximum amount of heat delivered to the tissue in the range of 1.5-6 MHz compared to the 8-14 MHz for maximal URF in FIG. 6a. The exponential in Eq. (8) shows that the frequency for maximal heat delivery decreases with increasing depth. Relationship between the heat energy delivered and the temperature achieved in the tissue, is established from experimental evidence, where the temperature also can be imaged as described below.

FIG. 7b shows an example simulation of the variation with depth of the locally delivered heat, for the transmitted ultrasound center frequency of 4 MHz. The same simulation parameters as in FIG. 6 are used. We note that for a given frequency the depth variation of the locally delivered heat is dominated by the exponential function in Eq. (8) and hence has a uniform decrease with much slower variation with depth compared to the URF. However, for a given depth there is a frequency that maximizes the UH as shown in FIG. 6b for a depth of 22 mm. As σₑ>0 and increases with frequency, the exponential in Eq. (8) implies that the frequency for maximal UH decreases with depth. For the liver or kidney the maximum UH is found at ˜1-1.5 MHz depending on depth This means that the UH can be obtained with much fewer focal regions per beam direction.

Hence, by varying the transmitted ultrasound center frequency one can vary between large URF with low UH, and vice versa. By selecting an intermediate frequency, one can similarly obtain a simultaneous defined URF and UH.

We define therapeutic hyperthermia as an increase in tissue temperature that has a marked additional therapeutic effect, both though increased blood flow that has significant effect on the therapy, increased diffusion of therapeutic agent, and direct thermal destruction of diseased tissue for especially high temperatures (˜60 deg). Non-therapeutic hyperthermia is a tissue heating, for example generated by an URF beam that does not produce marked therapeutic effects. By selecting the transmitted ultrasound frequency and scanning pattern, one can differentiate between URF without therapeutic heating of the tissue, and URF with therapeutic heating of the tissue.

Embodiments therefore also device to adjust the transmitted frequency, scanning pattern and rate so that one obtains a balanced simultaneous UH and URF effect. To increase Tₕ over a region R larger than determined by the beam width, one can for example scan the UH beam rapidly across the region R so that one for the heating observes an average intensity delivered to the region R. A method to measure temperature with ultrasound imaging is given in U.S. Pat. No. 8,038,616 and U.S. patent application Ser. No. 12/500,518 (2010/0036244), and the temperature can be imaged with MR imaging with known methods.

### Instrument

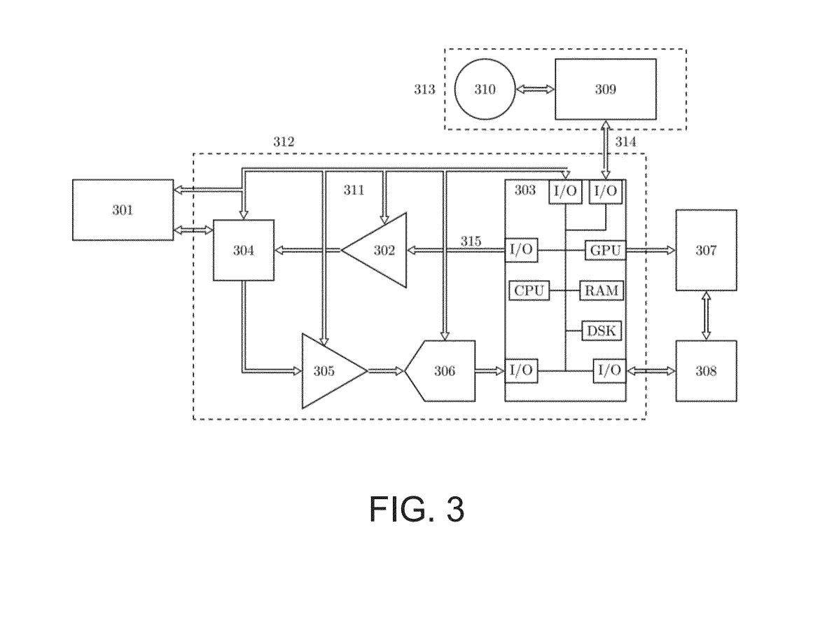

FIG. 3 shows an ultrasound instrument 312 operating according to the methods of embodiments. 301 shows an ultrasound transducer array structure adapted to transmit and focus ultrasound beams at least for the use of URF according to the methods described above, and potentially also in multiple frequency bands as required by different embodiments, for example to generate high MI beams, multiband ultrasound imaging, and also high UH of the tissue as discussed above. The arrays can be composed of a single wide band array that allows transmit of the different frequency bands from the same array, or they can be separate arrays for some frequency bands. Separate arrays can be mounted together to have at least part of the radiation surfaces common, for example as described in U.S. Pat. Nos. 6,645,150, 7,727,156 and 8,182,428.

The arrays can be arranged for electronic scanning of the ultrasound beams within an azimuth plane, where 3D scanning of the ultrasound beam is obtained by mechanical movement of the arrays for scanning of the azimuth plane in an elevation direction essentially normal to the azimuth plane. The arrays can also have a matrix structure for electronic 3D scanning of the ultrasound beams in both the azimuth and the elevation directions. One can also use annular arrays with full mechanical scanning of the beam direction within a 3D region. The annular array has some advantages for symmetric focusing of the beam and for low diffraction beams (Bessel beams).

The instrument 312 and the array structure 301 can mechanically be built into the same structure, for example a patient bed where the patient rests while being treated, or the bed of an imaging system such as with MR imaging, CT imaging, nuclear imaging, etc. In other embodiments, the array structure 301 can be connected to the instrument 312 via a flexible cable so that the array structure can be moved freely over the patients body by the operator, to position the array where one obtains best ultrasound access to the diseased tissue. In this case the array structure 301 can be mounted in a patient bed with the instrument by the side, or the array structure can be connected to a holder mechanism that allows fixation of a selected array position in relation to the patient. The array structure can also be given a shape that allows positioning of the array inside the patients body during treatment, for example in a trans rectal, trans gastric, trans vaginal, or trans esophageal position. The invention also encompasses array embodiments that are adapted to be used during surgery. With matrix arrays the array structure would typically contain sub-aperture electronics to reduce the number of cables between the array structure and the instrument.

302 of the instrument 312 shows a set of array transmit amplifiers that drive selected array elements of 301, potentially in different frequency bands to generate ultrasound beams in different frequency bands, for example for high URF, high MI, and high UH beams. The input signals to the array amplifiers 302 are given by the instrument processor 303 via an input-output (I/O) unit of the processor and the signal bus line 315. For use of at least parts of the arrays for ultrasound imaging, the imaging signal lines to said parts of the array are coupled to an optional transmit/receive switch 304 which after transmission couples said imaging signal lines to a set of receiver amplifiers 305 where the outputs are digitized in 306 and the digital signals are transmitted to the instrument processor 303 to construct the image, for example a 3D ultrasound image, that is displayed on the instrument display screen 307. The instrument processor 303 and display screen 307 interacts with a human interface unit 308 that allows the instrument operator to interact with, and control the instrument. The human interface unit is typically composed of a keyboard with alphanumeric and control keys, programmable keys, devices for pointing and interacting with the display screen, etc, according to well-known methods.

The instrument processor 303 can typically be of a personal computer (PC) type with one or more parallel Central Processing Units (CPUs) with typically multiple cores per CPU. The CPU addresses random access memory (RAM) and disk memory (DISK) in the form of mechanically rotating disks or solid state disk (SSD) over the processor bus. The CPU also communicates with input-output (I/O) devices that can be specially designed for example to generate transmit pulse sequences for the transmit amplifiers 302 and receive digital signals from the AD converters 306, communicate with the user interface 308 and interact with the control buses 311 and 314, that allows communication between the processor 303 and the other components of the instrument to set up the other components for the operation selected for a specific operation of the instrument. The image for the display is typically generated with one or more Graphics Processor Unit (GPU) based on data and instructions provided by the CPU. Each GPU contains more than hundred parallel cores for display of images on 307. The GPUs are also useful for advanced mathematical processing of algorithms according to embodiments, for example for noise suppression, to find the boundaries of the diseased tissue in the images, and nonlinear wave propagation simulation, for example as described in the Appendix, to determine the frequencies, focusing and scanning pattern of the treatment beams during treatment planning.

With this embodiment of the instrument 312 described in FIG. 3 one can hence obtain a 3D image of the diseased tissue as exemplified by a 3D ultrasound image of a tumor in FIG. 1, to be used to guide the treatment insonification of the diseased tissue by the instrument as described above. The scanning of the ultrasound beams for the different treatment applications (high URF, high MI, high UH) and the time sequence of the scanning, is then selected by the instrument processor 303 based on the treatment set-up planned and selected by the instrument operator. The instrument processor can be set up to analyze 3D images to automatically determine the boundaries of the diseased tissue, or the boundaries can be defined by the instrument operator using pointing devices to the image on the screen, or a combination of both.

Ultrasound imaging can for example be done with the high frequency band of the array assembly 301 for example centered around ˜8 MHz for tumors in the breast, the prostate, and similar. The URF beam can then be emitted in the same band from the same array, and a single band transducer array, for example a linear array, can then be used both for the imaging and the URF treatment. One preferably also wants to use dual band ultrasound pulses for the imaging, as described in U.S. Pat. No. 8,038,616 and U.S. patent application Ser. No. 12/500,518 (2010/0036244), where one for example could use a high frequency (HF) band centered around ˜8 MHz and a low frequency (LF1) band centered around ˜0.8 MHz according to the cited patent and patent application. The LF1 band is then also useful for generating high MI beams according the invention, but it is also interesting to use even lower frequency band LF2 for the MI beam, say ˜100-400 kHz. Such a 3-band array can be designed according to U.S. Pat. No. 8,182,428.

The frequencies for imaging, URF, and UH are determined by the depth of the diseased tissue and the ultrasound power extinction in the surrounding and diseased tissues. For deeper locations of the diseased tissue, for example a tumor in the liver or the kidneys, one would typically use a HF-band centered in the range ˜3-5 MHz for the imaging and the URF. The frequencies for maximal UH are lower ˜1 MHz. With dual band imaging, the LF1 band would typically be centered around ˜0.3 MHz, which also is a convenient band to generate the high MI pulses for these organs, although it can be advantageous to go to even lower frequencies for the high MI pulses for deep tissues. In embodiments of the instrumentation, the instrument processor 303 takes as input from operator and/or image the type and depth of the diseased and the surrounding tissue, and presents on this basis proposals for the frequencies to be used for the high URF treatment beams, and potentially also frequencies for high MI treatment beams and high UH treatment beams. The operator would typically do the final selection of frequencies depending on the operating bands of ultrasound transducer arrays available, and special patient considerations like fat in the tissue.

In other embodiments of the invention, the ultrasound imaging can be substituted with or accompanied by other imaging modalities, for example but not limited to magnetic resonance (MR) imaging, nuclear imaging, X-ray imaging, photo-acoustic imaging, or optical imaging, as described above. Such optional imaging is included as 313 in FIG. 3, where 309 illustrates an added imaging processor communicating with the treatment instrument processor 303 and further to the display 307 and human interface 308 over the bus 314. In some embodiments, the processor 303 can also operate as a processor for the imaging modality 313. 310 illustrates the sensors that interacts with the patient, where the type of sensors are defined by the imaging modality. For photo-acoustic imaging 310 could be the light (laser) source, while sensing of the ultrasound is done by ultrasound transducer elements of 301, where the processor 303 sets up the ultrasound receiver beam forming for photo-acoustic imaging. It is also beneficial to use a combination of imaging modalities in the instrument, for example a combination of ultrasound, photo-acoustic, MR, and nuclear imaging, for improved imaging and information extraction regarding the diseased tissue. For example can combined imaging modalities be used to estimate ultrasound absorption and extinction coefficients of the diseased and surrounding tissue, to be used in the determination of treatment frequencies for high URF and high UH therapeutic beams. MR imaging can be used to monitor tissue temperature. The imaging component and the ultrasound treatment scanning component is then set up and calibrated so that the coordinates of the ultrasound therapy scanning can be defined from the images. In some embodiments the Ultrasound instrument 312 and transducer unit 301 can be built into the same unit as 313, for example the patient bed of a MR, CT, or PET imaging system.

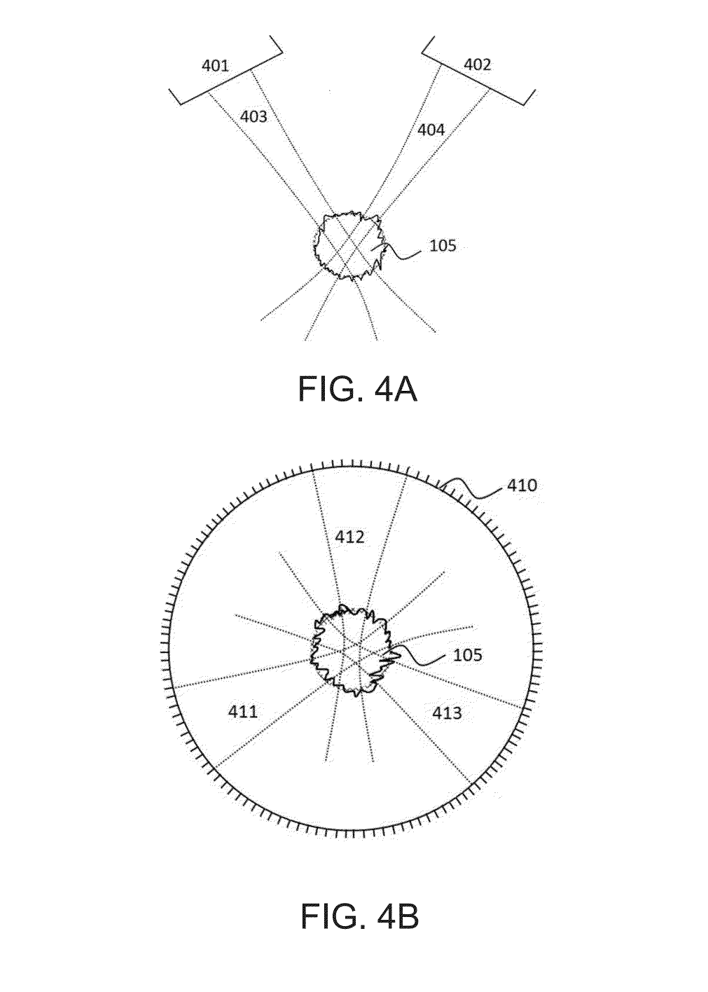

FIGS. 4a and 4b illustrates example embodiments of ultrasound transducers to irradiate the diseased tissue 105 with ultrasound beams from different directions, in a sequence or simultaneously. FIG. 4a shows by way of example two linear arrays 401 and 402 that insonifies the diseased tissue 105 with two beams 403 and 404 from different directions. It is also interesting to use out of plane arrays that irradiates the diseased tissue with a beam crossing the paper plane of the Figure. With the linear arrays, the beams can be scanned electronically in the azimuth plane, and moved mechanically in the elevation direction, to scan the beams in a 3D manner across the diseased tissue. A similar arrangement can also be obtained with phased arrays. With matrix arrays the elevation beam scanning can be obtained with electronic beam steering.

FIG. 4b shows as 410 a ring array that allows free selection of for example three ultrasound beams 411, 412, and 413 that insonifies the diseased tissue 105 from different directions. The beams can be scanned electronically in an azimuth plane. Beam scanning in the elevation direction can be obtained by mechanical movement of the ring array, or electronically with division of the array elements in the elevation direction. The ring array is for example useful for treatment of breast cancer.

Instruments and associated operating methods according to embodiments are operable so that they through selection of transmit parameters can be used for sequential or simultaneous transmission of

i) High URF beams at relatively high frequency (e.g. ˜3.5-15 MHz) with a MI that has a magnitude that results in particle breakage below a predetermined percentage of the particles over a predetermined period of time, to push therapeutic agent in the direction of the beam, and
ii) An ultrasound beams at low frequency (e.g. ˜0.3 MHz) with MI that results in the breakage of a minimum percentage of the particles over a predetermined period of time breakage of particles and stimulation of transport of therapeutic agent across membranes, or healing of the border zone of an infarction, and
iii) High UH beams at an intermediate frequency (e.g. ˜1-4 MHz) for heating of the diseased tissue for example to stimulate flow of blood with therapeutic agent, oxygen and nutrition to the diseased tissue, manipulate thermally sensitive particles, or direct thermal destruction of tissue.

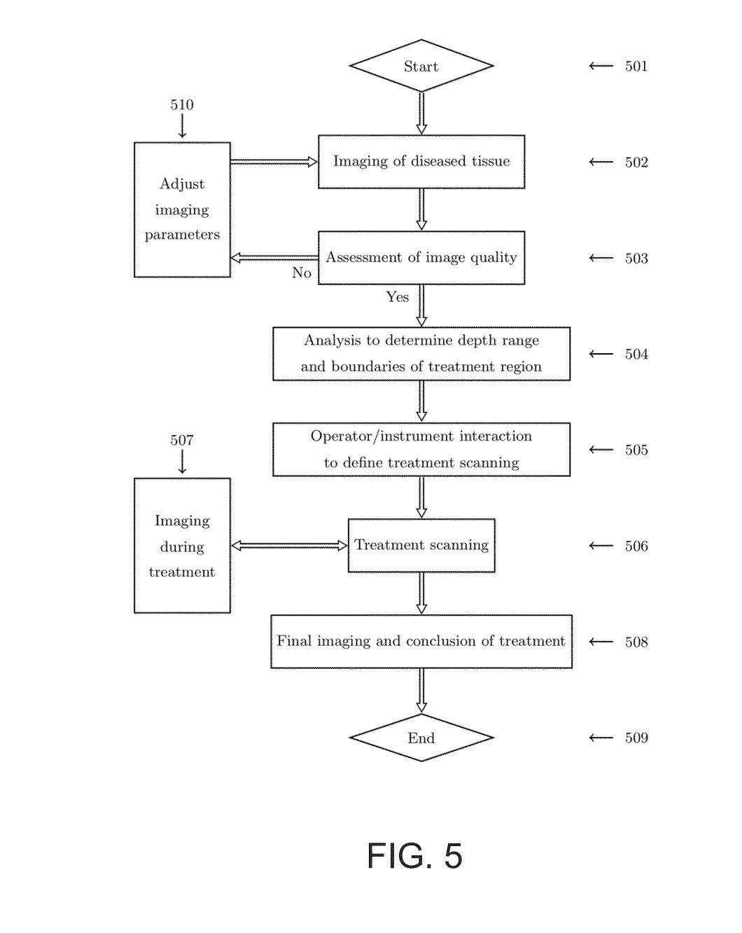

A typical use of the instrument is composed of several different operations in a sequence that is controlled by the instrument processor 303. Planning and selection of the operating sequences can for example be done as illustrated in the block diagram of FIG. 5. Upon start of the operation, 501, the instrument obtains an image of the diseased and surrounding tissue illustrated by block 502, using by example the imaging modalities described above, typically a 3D image, for example as illustrated in FIG. 1. The image quality is assessed (503), and with inadequate image quality, imaging parameters and potentially also patient position are adjusted and contrast agents are potentially added (510), and a new image is generated. For ultrasound imaging, typical imaging parameters are ultrasound frequency and position of the ultrasound probe. Other imaging modalities allow changes in other imaging parameters to improve image quality. After obtaining adequate quality of the image, the instrument is switched to an image analysis mode (504), where the outer boundaries of the diseased tissue, particularly the depth range, are determined. For some tumors there are tumor cells that infiltrate the diseased tissue outside the compact part of the tumor, and dependent of the treatment strategy one can include a region outside the compact tumor in the region of diseased tissue, and hence define a treatment region outside the compact tumor.

The instrument is then switched to a treatment beam planning mode (505), where based on an input of type of diseased and surrounding tissue the treatment beam types (i.e. high URF, high MI, high UH) and sequence and potential simultaneity of treatment beam types are determined in interaction with the operator. Instruments according to the invention can therefore be set up in several operating modes of combining high frequency, high URF and low MI beams, and low frequency, high MI and low URF beams, both simultaneously and sequentially in any order, for example but not limited to:

1. First insonify the diseased tissue with high URF beams to transport therapeutic agent (nanoparticles or molecules) from the capillaries and deep into the interstitium, followed by lower frequency, high MI beams (as discussed above) for breakage of particles to release therapeutic agent in the interstitium and also stimulate transport of therapeutic agent across cell membranes, followed by high frequency URF beams to further transport therapeutic agent towards and into the cells, or
2. When some of the particles are ultrasound sensitive phase changing nanoparticles, it can be useful in sequence 1 to apply a UH beam scan after the first URF beam scan and before the MI beam scan, to generate gas bubbles that are manipulated with the MI beam scan, for example to break other nanoparticles and stimulate transport of therapeutic agent across cell membranes, or
3. First insonify the diseased tissue with beams that have a MI that is known generate cavitation of microbubbles to open the blood brain barrier, followed by therapeutic sequence 1 or 2 above, or
4. First insonify the diseased tissue to increase blood flow and therapeutic agent to the diseased tissue as a result of UH, followed by therapeutic sequence 1 or 2 or 3 above, or
5. In part or the whole of the sequences, the instrument can be set up with simultaneous transmission and scanning of different types of treatment beams, for example high MI beams and high URF beams for simultaneous high MI opening of biological membranes and high URF to force the transport of agents across the membranes.

From an input of the type of diseased and surrounding tissue (e.g. breast tissue, prostate tissue, myocardium, etc.), and the depth range of the selected treatment region, for example by direct input form the operator or through the processors analysis of images, or a combination of both, the processor 303 calculates a choice of transmit parameters such as transmit aperture, transmit focus, transmit pressure and frequency, (e.g. ˜10 MHz for the set-up in FIG. 6a) for high URF, and potentially also a different choice of transmit parameters for high UH (e.g. ˜3 MHz for the set-up in FIG. 6b), or a good choice of transmit parameters for combined URF and UH beam scanning. The beam scanning parameters such as beam scanning region and beam scanning rate are also selected.

The transmit parameters can for example be obtained from pre-established multidimensional data stored in the processor, for example through direct look-up of nearest values in a table or through a functional adaptation to experimental data, for example obtained through interpolation between experimental data in a multidimensional table. The table can for example be obtained through multiple experiments with potential support of computer simulations including simulations of nonlinear wave propagation and subsequent URF and UH, for example as described in the Appendix, where the acoustic tissue parameters can be obtained from multiple experiments as described for the table above. According to an embodiment of the invention, the processor creates the table through computer learning of the results of multiple procedures carried through by the instrument, and potentially also other instruments connected in a network. Data or tables recommended by a certifying body can be established in this way.

The selection of the treatment beams and scanning pattern is done by the instrument operator assisted by algorithms run by the instrument processor 303 operating as described above.

The instrument is then set into treatment scanning mode (506), where the treatment beams and scanning pattern sequences as planned during 505, are generated by the instrument. During the treatment scanning one can optionally do imaging as in 507 for example to make sure that the diseased tissue does not move away from its original location in the image and hence also the treatment beams, and also to observe tissue parameters modified by the treatment such as increased tissue temperature or cavitation within the tissue. For ultrasound imaging, one might have to interrupt the treatment beams during the imaging, especially beams for URF and UH as they use frequencies close to that used for imaging and hence can interfere with the imaging. With MR imaging one can have electromagnetic interference from the treatment beams to the MR pick-up signals. Synchronizing treatment pulses with MR pick-up signals reduces this interference. Optical, X-ray, and nuclear imaging gets less interference from the treatment pulses. After the time set in the treatment planning phase 505, or through direct interference from the operator, the treatment pulses end. It is generally recommended that a final imaging of the treated tissue is done as in 508 after the treatment, for example to assess end stage tissue parameters such as temperature and cavitation, and verify that the diseased tissue has not moved out of the region of the treatment beam scanning. The procedure finally ends in its conclusion in 509.

Whilst the method described with reference to FIG. 5 relies on ‘pilot’ images to determine the depth of the diseased tissue as a basis for the transmit parameters to be used, the depth of the tissue to be treated within a body may be known, say from earlier examination, and such pilot images may therefore not be required.

Thus, while there have shown and described and pointed out fundamental novel features of the invention as applied to preferred embodiments thereof, it will be understood that various omissions and substitutions and changes in the form and details of the devices illustrated, and in their operation, may be made by those skilled in the art without departing from the spirit of the invention. It is also expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

### APPENDIX

### Simulation of URF and UH

Nonlinear elasticity of tissue produces a pressure dependent propagation velocity of ultrasound waves as

c(p)=c₀(1+βₚp) (A1)

where βₚ is the nonlinearity parameter of the tissue elasticity, p is the local pressure, and c₀˜1.45 mm/μsec is the propagation velocity for low pressure amplitudes. The nonlinear tissue elasticity produces the following equation for nonlinear wave propagation

$${▽}^{2} p ( \underline{r} , t ) - \frac{1}{{c}_{0}^{2}} \frac{{\partial }^{2} p ( \underline{r} , t )}{\partial {t}^{2}} - {h}_{ɛ} ( t ) {⊗}_{t} \frac{1}{{c}_{0}^{2}} \frac{\partial p ( \underline{r} , t )}{\partial t} + \frac{{β}_{p}}{{c}_{0}^{2}} \frac{{\partial }^{2} {p ( \underline{r} , t )}^{2}}{\partial {t}^{2}} = 0 \qquad ( A2 )$$

where p(r,t) is the pressure at spatial location r and time t. The last term represents the effect of nonlinear elasticity in the tissue. The convolution kernel hₑ in the 3^{rd} term represents the extinction of energy from the forward propagating wave due to thermal absorption and scattering. For numerical simulation of the nonlinear wave propagation it is convenient to use what is termed the retarded time coordinate

τ=t−z/c₀p(z,rₙ,t)→\hat{p}(z,rₙ,τ) (A3)

where z is the coordinate along the beam axis and rₙ=xeₓ+ye_{y}=(x,y) represents the coordinate vector normal to the beam axis with the orthogonal unit vectors eₓ along the x-axis and e_{y} along the y-axis. The retarded time constant transforms Eq. (A2) to

$$\frac{{\partial }^{2} {p}^{⋒}}{\partial {z}^{2}} - \frac{2}{{c}_{0}} \frac{{\partial }^{2} {p}^{⋒}}{\partial z \partial τ} + {▽}_{n}^{2} {p}^{⋒} - {h}_{e} {⊗}_{t} \frac{1}{{c}_{0}^{2}} \frac{\partial {p}^{⋒}}{\partial t} + \frac{{β}_{p}}{{c}_{0}^{2}} \frac{{\partial }^{2} {{p}^{⋒}}^{2}}{\partial {τ}^{2}} = 0 \qquad ( A4 )$$

where the 3^{rd} term represents second order differentiation with respect to rₙ. In the so called KZK approximation the 1^{st} term is neglected, and Fourier transform in time then gives

$$\frac{\partial {p}^{⋒} ( \underline{r} , ω )}{\partial z} = \frac{{c}_{0}}{2 iω} {▽}_{n}^{2} {p}^{⋒} ( \underline{r} , ω ) - \frac{iω}{2 {c}_{0}} {H}_{e} ( ω ) {p}^{⋒} ( \underline{r} , ω ) + \frac{{iωβ}_{p}}{2 {c}_{0}} {p}^{⋒} ( \underline{r} , ω ) {⊗}_{ω} {p}^{⋒} ( \underline{r} , ω ) \\ {p}^{⋒} ( \underline{r} , ω ) {⊗}_{ω} {p}^{⋒} ( \underline{r} , ω ) = \frac{1}{2 π} \int d w {p}^{⋒} ( \underline{r} , ω - w ) {p}^{⋒} ( \underline{r} , ω ) \qquad ( A5 )$$

The convolution in frequency of the nonlinear term produces harmonic frequency components in the wave function that accumulates with propagation distance and amplitude of the pressure wave. Fourier transforming along the transversal space coordinate, allows integrating this equation with z, which gives us a method of simulation of the nonlinear wave propagation.

The imaginary part of Hₑ represents the extinction of intensity. Separating the 3^{rd} term in Eq. (A5) into real and imaginary parts provides:

$$\begin{matrix}\frac{iω}{2 {c}_{0}} {H}_{e} ( ω ) = \frac{iω}{2 {c}_{0}} {H}_{er} ( ω ) - \frac{ω}{2 {c}_{0}} {H}_{ei} ( ω ) \\ = \frac{iω}{2 {c}_{0}} {H}_{er} ( ω ) + \frac{1}{2} {σ}_{e} ( ω )\end{matrix} \qquad ( A6 )$$

where Hₑᵣ and Hₑᵢ are the real and imaginary components of Hₑ respectively, and σₑ is the extinction cross section as defined in Eq. (1). The extinction of intensity is a causal, physical process. Therefore Hₑᵣ=Hi{Hₑᵢ}, where Hi is the Hilbert transform.

A practical model for the extinction cross section over a limited band centered around ω₀ is

σₑ(ω)=σₐ(ω)+σₛ(ω)=σₐ₀((ω/ω₀)^{b}+σᵣ₀(ω/ω₀)^{a}) (A7)

σᵣ₀=σₛ₀/σₐ₀ is the ratio between the scattering and absorption component of the extinction cross section at ω=ω₀. Typically σᵣ₀˜0.05-0.2. A rule of thumb is that the extinction cross section is X dB/cmMHz relating to the pulse center frequency, where X typically is 0.5 dB/cmMHz.

In the aforementioned simulations σₐ₀ was determined as

$$\begin{matrix}{σ}_{e} ( {ω}_{0} ) = {σ}_{a 0} ( 1 + {σ}_{r 0 } ) \\ = \frac{10 X}{1 ge} \frac{{ω}_{0}}{2 π} {m}^{- 1}\end{matrix} \qquad ( A8 )$$

and use b=1.2, a=2, σᵣ₀=0.1 and X=50 10⁻⁶ dB/mHz for the simulations in FIG. 6 and FIG. 7.

A pulse U(ω,ω₀) can be assumed to be transmitted with angular frequencies ω in a band centered around the center frequency ω₀. The pulse can be given a spatial appodization A(r₀) across the transmit array aperture, where r₀ is the spatial coordinate of the array elements. To focus the beam onto a focus location rₜ, the transmit signals is given a focusing delay τ(r₀; rₜ) that can be calculated as

$$τ ( {\underline{r}}_{0} ; {\underline{r}}_{t} ) = \frac{1}{{c}_{0}} ( \sqrt{{r}_{i}^{2} - {r}_{0}^{2}} - {r}_{i} ) + {τ}_{abc} ( {\underline{r}}_{0} ; {\underline{r}}_{t} ) \qquad ( A9 )$$

where τ_{abc}(r₀; rₜ) is an aberration correction delay which need to be estimated in some way, for example as described in U.S. Pat. No. 6,485,423, U.S. Pat. No. 6,905,465, or U.S. Pat. No. 7,273,455. Even if correct correction delays are not known, it can be advantageous to add a random delay with a given amplitude. Such a random delay would defocus the transmit beam somewhat, but the beam would be less sensitive to further defocusing due to wave front aberrations in the tissue. The transmitted pressure field at the surface of the array would then be

Pₜ(r₀,ω;ω₀)=U(ω;ω₀)A(r₀)e^{-iωτ(r}^{₀}^{;r}^{ₜ}⁾ (A10)

This would be used as a starting point at z=0 for numerical simulation of Eq. (A5) which would give us the transmitted pressure beam as a function of the spatial coordinate r and the angular frequency ω. The ultrasound radiation force per unit volume at location r and averaged over the pulse length Tₚ, then takes the form

$$\begin{matrix}\overline{f} ( \underline{r} , {ω}_{0} ) = \frac{Δ F ( \underline{r} , {ω}_{0} )}{Δ V} \\ = \frac{1}{2 {πcZ}_{0} {T}_{p}} \int d {ωσ}_{e} ( ω ; {ω}_{0} ) {\vert P ( \underline{r} , ω ; {ω}_{0} ) \vert}^{2} \frac{N}{{m}^{3}}\end{matrix} \qquad ( A11 )$$

where Z₀ is the characteristic acoustic impedance of the tissue. As presented above, of interest is the URF acting on a fairly low viscosity fluid. Since the URF is proportional to the pressure amplitude squared, there is a strong advantage in focusing the beam to increase the URF. Of general interested is therefore the URF near the focus, i.e. r≈rₜ.

The heat delivered to a volume element ΔV=ΔzΔrₙ² at location r=(z,rₙ) within the beam is

$$\frac{Δ Q ( \underline{r} ; {ω}_{0} )}{Δ V} = \frac{1}{2 π {Z}_{0}} \int d {ωσ}_{a} ( ω ) {\vert P ( \underline{r} , ω ; {ω}_{0} ) \vert}^{2} \frac{Joule}{{m}^{3}} \qquad ( A12 )$$

As heat is rapidly transported to neighboring volumes with blood perfusion and thermal diffusion in the tissue, there is further interest in the heat energy delivered across the whole beam as a function of depth z, which takes the form

$$\frac{Δ Q ( z ; {ω}_{0} )}{Δ z} = \frac{1}{2 π {Z}_{0}} \int d {ωσ}_{a} ( ω ) \int {d}^{2} {r}_{n} {\vert P ( z , {\underline{r}}_{n} , ω ; {ω}_{0} ) \vert}^{2} \frac{Joule}{m} \qquad ( A13 )$$

1. An instrument suitable for ultrasound mediated transport of a therapeutic agent in a region of diseased tissue, comprising:
a transmitter adapted for transmitting at least one ultrasound radiation force beam, hereinafter referred to as URF beam, for insonifying at least a region of tissue, wherein at least one of a scanning rate for scanning the beam across a region of tissue, a transmit focus, a transmit aperture, and a transmit frequency of said URF beam is selectable,
a processor configured to calculate, based on an indication of a tissue type or of tissue types to be insonified and on an indication of a depth range within which ultrasound intensity and extinction of intensity is to be increased, at least one of the following transmit parameters for said at least one URF beam:
i) a number of URF beam transmit foci to be used;
ii) respective depths for one or more URF beam transmit foci;
iii) a URF beam transmit aperture for the URF beam transmit focus or respective URF beam transmit apertures for each of more than one URF beam transmit foci;
iv) a URF beam transmit frequency for a URF beam transmit focus or respective URF beam transmit frequencies for each of more than one URF beam transmit foci;
v) a URF beam transmit pressure for a URF beam transmit focus or respective a URF beam transmit pressures for each of more than one URF beam transmit focus;
vi) a URF beam scanning region; and
vii) a URF beam scanning rate, and
set up means for setting up said transmitter with the calculated transmit parameter or transmit parameters for said URF beam.

2. The instrument according to claim 1, where said transmitter comprise means for insonifying at least one region of tissue with URF beams from multiple directions at least one of sequentially and simultaneously.

3. The instrument according to claim 1, where said transmitter is adapted for transmitting a beam at a frequency less than ⅕ of the URF frequency and a mechanical index, hereinafter referred to as MI, of more than 0.5, sequentially or simultaneously with said URF beam.

4. The instrument according to claim 3, where
said transmitter is adapted for transmitting simultaneously or sequentially with said URF beam, an ultrasound heating, hereinafter referred to as UH, beam for therapeutic heating of tissue with at least one UH beam, where at least one of a scanning rate for scanning the beam across a region of tissue, a transmit focus, a transmit aperture and a transmit frequency of said UH beam is selectable, and
said processor is configured to calculate, based on an indication of a depth range within which the tissue is to be heated, at least one of the following transmit parameters for said UH beam:
i) a number of UH beam transmit foci to be used,
ii) respective depths for one or more UH beam transmit foci;
iii) a UH beam transmit aperture the UH beam transmit focus or respective UH beam transmit apertures for each of more than one UH beam transmit foci;
iv) a UH beam transmit frequency for a UH beam transmit focus or respective UH beam transmit frequencies for more than one UH beam transmit foci;
v) UH beam transmit pressures for each transmit focus, and
vi) a UH beam scanning region;
vii) a UH beam scanning rate; and
said set up means is adapted for setting up said transmitter with the calculated transmit parameter or transmit parameters for said UH beam.

5. The instrument according to claim 1, where
said transmitter is adapted for transmitting simultaneously or sequentially with said URF beam, an ultrasound heating, hereinafter referred to as UH, beam for therapeutic heating of tissue with at least one UH beam, where at least one of a scanning rate for scanning the beam across a region of tissue, a transmit focus, a transmit aperture and a transmit frequency of said UH beam is selectable, and
said processor is configured to calculate, based on an indication of a depth range within which the tissue is to be heated, at least one of the following transmit parameters for said UH beam:
i) a number of UH beam transmit foci to be used,
ii) respective depths for one or more UH beam transmit foci;
iii) a UH beam transmit aperture the UH beam transmit focus or respective UH beam transmit apertures for each of more than one UH beam transmit foci;
iv) a UH beam transmit frequency for a UH beam transmit focus or respective UH beam transmit frequencies for more than one UH beam transmit foci;
v) UH beam transmit pressures for each transmit focus, and
vi) a UH beam scanning region;
vii) a UH beam scanning rate; and
said set up means is adapted for setting up said transmitter with the calculated transmit parameter or transmit parameters for said UH beam.

6. The instrument according to claim 1, where said processor is configured to calculate the at least one of the transmit parameters based on stored data encoding the depth dependency of the at least one of the transmit parameters stored in said processor.

7. The instrument according to claim 1, where said processor is configured to calculate the at least one of the transmit parameters based on simulations of wave propagation in tissue.

8. The instrument according to claim 1, further comprising means for generating an image of the diseased tissue and means for defining the region of diseased tissue in the image, said processor being configured to calculate the at least one of the transmit parameters based on the defined location of the diseased tissue as input.

9. The instrument according to claim 8, further comprising means for imaging cavitation in tissue during insonification.

10. The instrument according to claim 9, where said transmitter and said scanning means are adapted for transmitting a beam at a frequency less than ⅕ of the URF frequency and an MI of more than 0.5, sequentially or simultaneously with said URF beam, the instrument further configured to increase MI to increase cavitation and/or to decrease MI to decrease cavitation.

11. The instrument according to claim 5, further comprising means for imaging temperature in the tissue during insonification, the instrument further configured to, based on temperature images of insonified tissue, select UH beam transmit parameters to obtain a tissue temperature close to or not exceeding a defined temperature.

12. The instrument according to claim 1, where said processor comprises set up means configured to support the instrument operator in planning a sequence of treatment scans, said treatment beam scans being composed of at least one of
i) an URF beam scan, and
ii) an high MI beam scan, and
iii) an UH beam scan
that are set up in a freely selectable order as one of a i) in a sequence, and ii) simultaneously, and iii) a combination of sequence and simultaneously, and
said set up means comprises means to set up the instrument to carry through said planned sequence of treatment scans.

13. The instrument according to claim 6, where said processor is configured to calculate the at least one of the transmit parameters based on computer learning from experimental data.

14. The instrument according to claim 7, where said processor is configured to calculate the at least one of the transmit parameters based on computer learning from experimental data.

15. A method for ultrasound mediated transport of a therapeutic agent in biological tissue, comprising:
calculating for a given depth range to be insonified, at least one of the following transmit parameters for use with at least one ultrasound radiation force beam, hereinafter referred to as URF beam:
i) number of URF beam transmit foci to be used;
ii) respective depths for one or more URF beam transmit foci;
iii) a URF beam transmit aperture for the URF beam transmit focus or respective URF beam transmit apertures for each of more than one URF beam transmit foci;
iv) a URF beam transmit frequency for a URF beam transmit focus or respective URF beam transmit frequencies for each of more than one URF beam transmit foci;
v) a URF beam transmit pressure for a URF beam transmit focus or respective a URF beam transmit pressures for each of more than one URF beam transmit focus;
vi) a URF beam scanning region; and
vii) a URF beam scanning rate;
transmitting said at least one URF beam using said at least one of the transmit parameters.

16. The method according to claim 15, where the ultrasound beam is transmitted from a wide aperture with strong focusing and where said focal region is scanned across diseased tissue.

17. The method according to claim 16, where multiple focal regions are applied sequentially along at least one beam direction.

18. The method according to claim 15, where said at least one URF beam is scanned so that a region of tissue is insonified with URF beams in multiple directions at least one of sequentially and simultaneously.

19. The method according to claim 15, further comprising the step transmitting a beam with MI>0.5, hereinafter referred to as high MI beam, at a frequency<⅕ of the frequency of the URF beam, one of sequentially and simultaneously with said URF beam, and scanning said at least one high MI beam across said region of the diseased tissue with selectable MI beam scanning region and MI beam scanning rate.

20. The method according to claim 15, further comprising:
calculating for a given depth range of diseased tissue, at least one of the following transmit parameters for at least one ultrasound heating beam, hereinafter referred to as UH beam, for therapeutic heating of at least a region of said diseased tissue:
i) number and depths of UH beam transmit foci, and
ii) UH beam transmit apertures for each focus, and
iii) UH beam transmit frequencies for each focus, and
iv) UH beam transmit pressures for each focus, and
v) UH beam scanning region and beam scanning rate,
transmitting sequentially or simultaneously with said URF beam, said at least one UH beam using said calculated UH beam transmit parameters, for therapeutic heating of at least a region of said tissue, and
scanning said at least one UH beam and foci across said region of the diseased tissue with selectable UH beam scanning region and UH beam scanning rate at least one of sequentially or simultaneously with the scanning of said URF beam.

21. An instrument suitable for ultrasound mediated transport of a therapeutic agent in a region of diseased tissue, comprising:
a transmitter and transducer array adapted for transmitting at least one ultrasound radiation force beam at a frequency>3 MHz, hereinafter referred to as URF beam, for insonifying at least a region of tissue, wherein at least one of a scanning rate for scanning the beam across a region of tissue, a transmit focus, a transmit aperture, and a transmit frequency of said URF beam is selectable, and
the transmitter and transducer array further adapted for transmitting at least one high mechanical index beam at a frequency<1 MHz, hereinafter referred to as MI beam, for insonifying at least a region of tissue, wherein at least one of a scanning rate for scanning the beam across a region of tissue, a transmit focus, a transmit aperture, and a transmit frequency of said MI beam is selectable.

22. The instrument according to claim 21, where said URF beam and MI beam are transmitted simultaneously.

23. The instrument according to claim 21, where said URF beam and MI beam are transmitted along the same beam direction.
